(19) United States
(12) Patent Application Publication
(10) Pub. No.: US 2024/0127912 A1
(43) Pub. Date: Apr. 18, 2024

(54) SYSTEMS AND METHODS FOR GENERATING CUSTOMIZABLE DIGITAL DATA STRUCTURES

(71) Applicant: Nuttall, Brown & Coutts, LLC, Cottonwood Heights, UT (US)

(72) Inventors: Tyler Brown, Cottonwood Heights, UT (US); Brian Coutts, Cottonwood Heights, UT (US); Scott Brown, Cottonwood Heights, UT (US); Eric Eliason, Cottonwood Heights, UT (US); Nate Obray, Cottonwood Heights, UT (US)

(21) Appl. No.: 18/487,847

(22) Filed: Oct. 16, 2023

Related U.S. Application Data
(60) Provisional application No. 63/417,076, filed on Oct. 18, 2022.

(51) Int. Cl. G16H 10/20; G06Q 40/08; G16H 10/60

(52) U.S. Cl. CPC G16H 10/20; G06Q 40/08; G16H 10/60

(57) ABSTRACT

Embodiments of the present disclosure include a method that accesses various portions of data related to an event in which a user was involved. The method further determines, for the event, which informatory elements related to the event meet at least a minimum significance threshold. The method next presents to the user, via a graphical user interface, one or more informational prompts associated with the event to obtain supporting information related to those informatory elements associated with the event that were determined to meet the minimum significance threshold. The method also generates a digital data structure that includes at least a portion of dynamically written language related to the event and further includes at least a portion of the supporting information received from the user in response to the informational prompts. Other corresponding systems and computer-readable media are provided.

### CROSS-REFERENCE TO RELATED APPLICATIONS

This application claims priority to and the benefit of U.S. Provisional Application No. 63/417,076, filed on Oct. 18, 2022, which application is incorporated by reference herein in its entirety.

### BACKGROUND

People are injured in accidents of all types every day. Many accident victims suffer physical injuries or damage to their property. Often, parties to an accident have insurance to (at least partially) cover the appurtenant damages including medical costs, lost wages, pain and suffering, and potential repair bills. However, there are times when an insurer does not want to cover a particular expense or may disagree about how much compensation is owed or which costs should be borne by the insurance company. In such cases, the individual(s) may need to seek legal help to obtain a recovery that is rightfully theirs. Seeking legal help, however, may be costly and time-consuming. Moreover, many people do not know whether they have a good case against an insurer and don't know where to start to find that type of information.

### BRIEF SUMMARY

The embodiments described herein are directed to systems and apparatuses that generate digital data structures based on aggregated data gleaned from a user's responses to specific informational prompts. In one scenario, a method is provided that includes accessing various portions of data related to an event in which a user was involved. The method further includes determining, for the event, which informatory elements related to the event meet at least a minimum significance threshold. The method next includes presenting to the user, via a graphical user interface, one or more informational prompts associated with the event to obtain supporting information related to those informatory elements associated with the event that were determined to meet the minimum significance threshold. The method also includes generating a digital data structure that includes at least a portion of dynamically written language related to the event and further includes at least a portion of the supporting information received from the user in response to the informational prompts.

In some embodiments, the informational prompts are dynamically generated and include at least one free-form answer prompt. In some examples, the user provides a free-form answer to the free-form answer prompt that is analyzed to obtain additional supporting information related to the informatory elements. In some cases, the informational prompts are dynamically generated and include at least one prompt that has multiple selectable options that are selectable via the graphical user interface.

In some examples, the user selects at least one of the selectable options in the dynamically generated prompt, and the selected options are analyzed to obtain additional supporting information related to the informatory elements. In some cases, the user is a party involved in an accident and/or a witness to the accident. In some embodiments, the user additionally provides, as supporting information, data structures in a local data store owned by the user, data structures that include text, image, audio or video files, personal healthcare records (PHRs), data structures that include financial records, and/or data structures stored in a decentralized Health Information Exchange.

In some cases, the informational prompts presented to the user are generated dynamically and are unique to the event in which the user was involved. In some embodiments, the dynamically generated informational prompts are generated using artificial intelligence. In some cases, the artificial intelligence is trained to determine which informatory elements related to the event meet the minimum significance threshold. In some examples, the artificial intelligence is trained to consider at least two of the elements meeting the minimum significance threshold in different manners. In some examples, the artificial intelligence is trained to alter the informational prompts presented to the user in the graphical user interface based on which supporting information is provided by the user.

A corresponding system may be provided that includes: at least one physical processor and physical memory comprising computer-executable instructions that, when executed by the physical processor, cause the physical processor to: access various portions of data related to an event in which a user was involved, determine, for the event, which informatory elements related to the event meet at least a minimum significance threshold, present to the user, via a graphical user interface, one or more informational prompts associated with the event to obtain supporting information related to those informatory elements associated with the event that were determined to meet the minimum significance threshold, and generate a digital data structure that includes at least a portion of dynamically written language related to the event and further includes at least a portion of the supporting information received from the user in response to the informational prompts.

In some cases, the digital data structure may be a case summary that includes an analysis of the case. In some examples, at least a portion of the supporting information is incorporated into the case summary. In some embodiments, the user interface allows the user to select which supporting information is to be included in the case summary. In some cases, the supporting information includes at least one of pictures, videos, text messages, email messages, or health records. In some cases, the supporting information is accessed from a mobile electronic device associated with the user. In some embodiments, the system may further train a machine learning model to determine, for the event, which informatory elements related to the event meet at least the minimum significance threshold.

A corresponding non-transitory computer-readable medium may also be provided that includes one or more computer-executable instructions that, when executed by at least one processor of a computing device, cause the computing device to: access various portions of data related to an event in which a user was involved, determine, for the event, which informatory elements related to the event meet at least a minimum significance threshold, present to the user, via a graphical user interface, one or more informational prompts associated with the event to obtain supporting information related to those informatory elements associated with the event that were determined to meet the minimum significance threshold, and generate a digital data structure that includes at least a portion of dynamically written language related to the event and further includes at least a portion of the supporting information received from the user in response to the informational prompts.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 illustrates an embodiment of a computing architecture in which the embodiments described herein may operate.

FIG. 2 illustrates an embodiment of a flow chart of a method for generating digital data structures based on aggregated data gleaned from a user's responses to specific questions.

FIGS. 3A-3D illustrate embodiments of a graphical user interface in which free form or multiple-option selection questions are provided.

FIG. 4 illustrates an embodiment showing different types of supporting information that may be provided by a user.

FIG. 5 illustrates an embodiment of an artificial intelligence computing architecture in which embodiments described herein may operate.

### DETAILED DESCRIPTION OF EXEMPLARY EMBODIMENTS

The present disclosure is generally directed to systems and methods for generating customized digital data structures. These customized digital data structures may include written documents such as personal injury case summaries, settlement demand letters, or other types of data structures. In the past, such documents were assembled manually, and were often limited to the understanding of the individual or team preparing the documents. In the case of pro se individuals, for example, that were not represented by an attorney, those individuals often attempted to prepare case summaries or demand letters with little knowledge of what to include in the letters. More specifically, they had little or no knowledge as to the things that would materially help their cases with opposing parties such as insurance companies.

Accordingly, pro se individuals or even attorneys with working knowledge in the field of personal injury may unintentionally omit items that are helpful to personal injury claims that are outlined in the case summary. Moreover, in many such cases, the amount of information associated with an event, such as a car accident, may include thousands of pages of material (or more), potentially including videos, images, text messages, emails, or other materials. In such scenarios, a single person or even a whole team of people may be unable to analyze all of that information and assimilate the pertinent portions into a case summary. Still further, even if such information were assembled, it would still be performed subject to the biases and potential lack of knowledge of the individual(s) preparing the summary, especially in the case of pro se individuals.

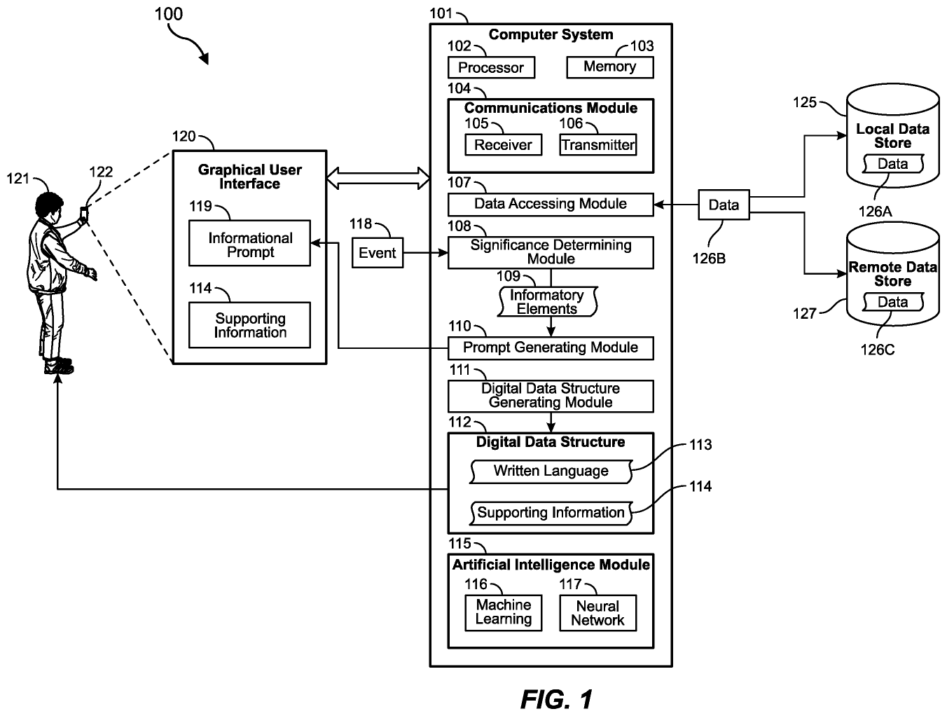

As will be explained below and with further regard to FIGS. 1-5, the embodiments described herein may provide methods for generating customized digital data structures including personal injury case summaries. A “case summary,” as the term is used herein, may refer to a document that summarizes facts relating to an accident (e.g., a car accident, bike accident, a slip and fall accident, a products liability injury, etc.), summarizes the accident victim's requests or demands, and potentially includes corroborating information. These personal injury case summaries may include many different types of information, including written paragraphs describing an incident, photographs or videos showing a given scene or the aftermath of an event (e.g., at an intersection), medical records, including statements, bills, or doctor notes, interviews conducted with witnesses, hospital records, or other supporting information.

Some of this descriptive and supporting information may be more pertinent to or more significant for a given event than other information. For instance, elements that support an injury claim or elements that reduce an individual's liability or negligence in a situation may be more valuable to include in the case summary. Knowing which elements to include in any given scenario is very difficult and tends to be highly subjective. Many pro se individuals do not know how to identify this information and, in the case of attorneys, the attorneys may now know where or how to find all of the pertinent information that may be beneficial in building an effective set of claims.

In one embodiment, a computer-implemented method may be provided that is directed to facilitating knowledgeable pro se handling of personal injury claims through automatically generated case summaries and/or settlement demand letters. This method may be carried out in the computing environment 100 of FIG. 1. For example, FIG. 1 illustrates a computing environment 100 in which specified types of data structures are generated automatically after receiving multiple replies to informational prompts from a user. FIG. 1 includes various electronic components and elements including a computer system 101 that is used, alone or in combination with other computer systems, to perform associated tasks. The computer system 101 may be substantially any type of computer system including a local computer system or a distributed (e.g., cloud) computer system. The computer system 101 includes at least one processor 102 and at least some system memory 103. The computer system 101 includes program modules for performing a variety of different functions. The program modules may be hardware-based, software-based, or may include a combination of hardware and software. Each program module uses computing hardware and/or software to perform specified functions, including those described herein below.

In some cases, the communications module 104 is configured to communicate with other computer systems. The communications module 104 includes substantially any wired or wireless communication means that can receive and/or transmit data to or from other computer systems. These communication means include, for example, hardware radios such as a hardware-based receiver 105, a hardware-based transmitter 106, or a combined hardware-based transceiver capable of both receiving and transmitting data. The radios may be WIFI radios, cellular radios, Bluetooth radios, global positioning system (GPS) radios, or other types of radios. The communications module 104 is configured to interact with databases, mobile computing devices (such as mobile phones or tablets), embedded computing systems, or other types of computing systems.

The computer system 101 further includes a data accessing module 107. The data accessing module 107 may be configured to access different types of data (e.g., 126A, 126B, and/or 126C) stored in local or remote data stores. For instance, the data accessing module 107 may be configured to access data 126A stored in a local data store 125 (e.g., on a person's phone, laptop, personal computer, or tablet). Additionally or alternatively, the data accessing module 107 may be configured to access data 126B from remote data store 127 (e.g., from a cloud platform, remote database, or other internet-accessible data store). The data may be combined (e.g., in data 126B) or may be accessed separately by the data accessing module 107.

The computer system 101 may further include a significance determining module 107. As noted above, not all information related to an accident or other event may be relevant or significant. Some facts or opinions may have no bearing on who was at fault, what the conditions were that led to the accident, or how coverage should be applied. The significance determining module 108 may be configured to analyze the data 126B and determine which informatory elements 109 from the data are significant. The data may be said to be “significant” or relevant to a given event if that data meets a minimum significance threshold.

To determine this minimum significance threshold, the significance determining module 108 may implement machine learning, neural networks, or other artificial intelligence algorithms to review past case summaries and/or demand letters, identify the results of those letters, and learn, in an iterative process, which details, which facts, or which opinions led to the most optimal results for the accident victim. During this process, different factors may be assigned to different elements of a case summary, and each piece of information from a user may be weighted as helping that factor or hindering that factor, with the goal being the highest possible settlement amount (or other favorable settlement terms). As will be explained further below, machine learning models may be trained to assign these weights to specific factors and determine which informatory elements 109 are the most significant in a given case and which data elements would be most valuable to include in that case summary.

Computer system 101 further includes a prompt generating module 110. The prompt generating module 110 may be configured to generate informational prompts 119 that are presented to a user 121 via a graphical user interface 120 (or “GUI 120”) herein. The informational prompts 119 may include questions that are presented to the user 121 that are intended to elicit certain types of information. At least in some cases, the informational prompts 119 may be unique for each event 118 or may be tailored to the event. The event 118 may be substantially any event against which an insurance claim may be filed. Such events may include automobile accidents, bicycle or motorcycle accidents, product liability injuries, loss of bodily functions, loss of life, or other occurrences that may lead to an insurance claim. Such claims typically require some level of substantiation. For instance, if a user is claiming some type of bodily injury, medical records may be used to substantiate that claim. Other types of substantiating information may include pictures, videos, text messages, emails, or other information that may be used to substantiate injury or loss claims. The informational prompts 119 may be designed and crafted to acquire information related to such an event.

The user 121 may respond to the informational prompts 119 in a variety of ways. For instance, in some cases, the informational prompts 119 may be free form, open-ended questions. The user 121 may use their mobile phone 122 or other electronic device to provide answers in free form prose. Additionally or alternatively, the informational prompts 119 may include more direct that are intended to elicit a more precise answer. In some cases, the informational prompt 119 may be a yes/no or true/false question that has a single answer. In other cases, the information prompt 119 may be a multiple-choice answer that offers a limited number of available answers. The prompt generating module 110 may combine these different styles of prompts to gather the types of information needed by the system to generate specific digital data structures including case summaries or settlement demand letters.

The digital data structure generating module 111 may be configured to generate these digital data structures 112. The digital data structures 112 may include multiple different divisions or sections, each of which may have a specific purpose as part of a case summary or other document. In some cases, the digital data structure 112 may include at least one section having written language 113, as well as various additional types of supporting information 114. This supporting information may include pictures of the event 118 (or its aftermath), videos of the event (e.g., dashcam videos or traffic light videos), emails or text messages between the accident victim and other victims, or between the accident victim and the insurance company, etc., health records of the accident victim showing which types of medical care were received, financial records showing financial losses resulting from the accident, information stored in a decentralized data exchange (e.g., health or financial data accessible to authenticated users), or other types of supporting information 114.

Still further, the computer system 101 in computing environment 100 may include an artificial intelligence module 115. The artificial intelligence module 115 may include machine learning modules 116, neural networks 117, or other artificial intelligence implementations. These modules, whether alone or in combination, may be implemented by the computer system 101 to perform many of the above-described tasks. For instance, the artificial intelligence (AI) module 115 may be configured to determine the significance of certain elements with relation to an event 118 by reviewing thousands or millions of previously filed case summaries and learning which elements were most significant in coming to an optimal case resolution (e.g., a maximum settlement amount).

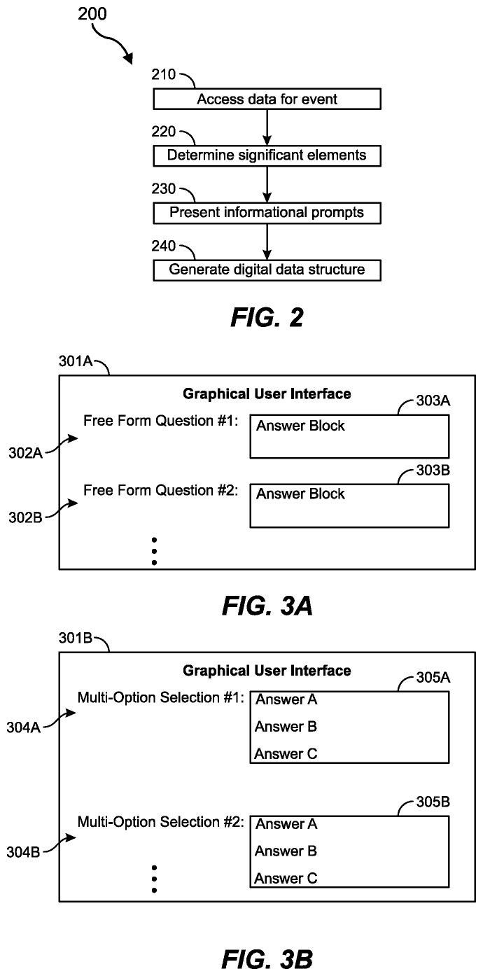

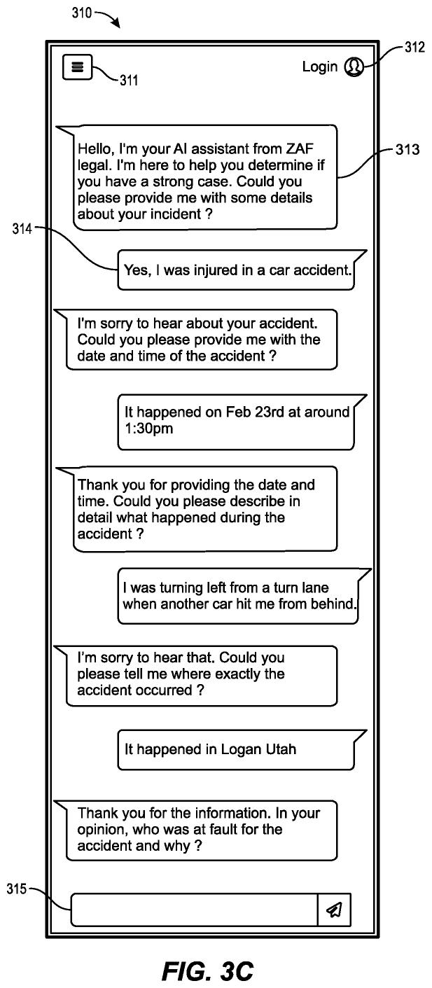

Additionally or alternatively, the AI module 115 may be configured to generate informational prompts 119. The AI module 115 may similarly learn from past informational prompts which prompts are most effective at eliciting the desired information (e.g., the information with the highest level of significance). The AI module 115 may then generate informational prompts 119 based on this learning, thereby focusing the user 121 on the most important questions and making the best use of the user's time and resources. Still further, the AI module 111 may be implemented to help generate the digital data structures 112, including optimizing the written language portions (e.g., 113), and helping to select the best supporting information 114. Thus, in this manner, it can be seen that the AI module 115 may perform or at least help with performing many of the tasks or functions described herein. These embodiments will be described in further detail below with reference to method 200 of FIG. 2 and with reference to FIGS. 3-5.

FIG. 2 is a flow diagram of an exemplary computer-implemented method 200 for generating digital data structures based on aggregated data gleaned from a user's responses to specific informational prompts. The steps shown in FIG. 2 may be performed by any suitable computer-executable code and/or computing system, including the systems illustrated in FIG. 1. In one example, each of the steps shown in FIG. 2 may represent an algorithm whose structure includes and/or is represented by multiple sub-steps, examples of which will be provided in greater detail below.

Method 200 includes, at step 210, accessing various portions of data (e.g., 126B of FIG. 1) related to an event 118 in which a user 121 was involved. The method 200 next includes, at step 220, determining, for the event 118, which informatory elements 109 related to the event meet at least a minimum significance threshold. Still further, method 200 includes, at step 230, presenting to the user 121, via a graphical user interface 120, one or more informational prompts 119 associated with the event 118 to obtain supporting information 114 related to those informatory elements associated with the event that were determined to meet the minimum significance threshold. Method 200 also includes, at step 240, generating a digital data structure 112 that includes at least a portion of dynamically written language 113 related to the event and further includes at least a portion of the supporting information 114 received from the user 121 in response to the informational prompts 119.

As noted above, the data accessing module 107 of FIG. 1 may be configured to access data (e.g., 126A, 126B, 126C, or other data) located in local and/or remote data stores 125 or 127, respectively. This data, which may include many different kinds of information related to an event 118, may then be accessed by the significance determining module 108 to determine a minimum level of significance. Because not all information related to an event 118 may be helpful in creating a positive result for a user (e.g., an accident victim), the significance determining module 108 may be implemented to identify those types of information that are valuable in generating a digital data structure that can produce a positive outcome for the user 121. In doing so, the significance determining module 108 may access many past case summaries, demand settlement letters, settlement results, and/or litigation results related to those case summaries or demand letters.

Upon accessing these summaries and results, the significance determining module 108 may perform iterative analyses (e.g., using machine learning or AI algorithms) to identify cases with positive outcomes and then further identify which language or which types of information were sought and provided in those cases. Upon looking at many different case summaries and resultant outcomes, the systems herein may determine the relative significance of certain pieces of information (e.g., location, time of day, parties involved, current weather, relative condition of traffic lights or signs, speed of the vehicles involved, whether substances were being used by the accident victims, whether the victims had been involved in previous accidents, or other information).

In some cases, the significance determining module 108 or the AI module 115 may assign weights to each type of information indicating their relative importance in including that information in a case summary or demand letter. In such cases, an informatory element 109 may need to meet at least a minimum weight or a minimum level of significance in order to be used by the system to generate an informational prompt. Information that does not meet this significance threshold is not sought in the first place and may be omitted when generating informational prompts 119. Accordingly, in this manner, the functionality of the computer system 101 may be improved by avoiding the expenditure of processor, memory, or other computing resources on generating prompts when the informatory significance of an element is below the established threshold.

Once the minimum significance threshold has been established, the prompt generating module 110 may generate various free form, multiple-choice, true/false, or other informational prompts 119 based on those informatory elements 109 that met the minimum significance threshold. The prompt generating module 110 may, at least in some cases, begin with free form questions and, after receiving a number of replies, may analyze the responses to determine whether the desired information has been provided in the user's replies. If not, the prompt generating module 110 may opt to change to more specific, multiple-choice or true/false or yes/no questions.

Then, based on the replies to these questions, the prompt generating module 110 may return to free form prompts or other types of questions. Thus, throughout the course of a prompt-and-answer session with a user, the prompt generating module 110 may switch between different types of prompts and may also switch between different questions that are substantively different (i.e., the questions have different subject matter and seek different information). In this manner, the prompt generating module 110 may dynamically change both the style or type of prompt as well as the substance of the prompt in order to optimally elicit a desired response. This dynamic updating may occur in response to the AI module 115 learning new techniques, new prompts, or new question styles.

Upon receiving certain expected answers, the prompt generating module 110 may transition back to free form prompts or may transition to a different type of prompt. The prompt generating module 110 may thus analyze received answers and, based on the answers, may dynamically switch from generating one type of prompt to another type. This process may be continually performed and continually updated while the user 121 is providing replies via the graphical user interface 120. In this manner, the prompt generating module 110 may dynamically select which type of prompt 119 is best to elicit a desired piece of information and may generate and present that prompt to the user on the graphical user interface 120. In some cases, the AI module 115 may be implemented to determine which type of informational prompt 119 would be best for any given piece of supporting information and may further be used to craft the wording used in the selected prompt.

Upon collecting the desired supporting information 114 from the user 121 via the informational prompts 119, the digital data structure generating module 111 may be implemented to create a digital data structure 112 that includes dynamically written language 113 related to the event 118 and may also include at least some of the supporting information 114 received from the user 121 in response to the informational prompts 119. The written language 113 may include language that outlines a case, including a background, description of the accident, description of the parties involved, an indication of demands, and other information appearing in a case summary. The written language may include human-readable prose divided into sentences and paragraphs, written cogently and competently enough to be submitted to a judge or tribunal. The written language 113 may be created using a generative pretrained transformer (GPT) model or other AI-based model designed to write prose in a specific language.

Each sentence or paragraph may be supported or documented using the supporting information 114 provided by the user 121 (or gleaned from other sources), including pictures, texts, emails, or other information relevant to the written language 113 in the digital data structure 112. The resulting digital data structure 112 may comprise a fully drafted case summary or settlement demand letter, with written notations and supporting evidence or other information. This digital data structure may be a word processing document, a spreadsheet document, a slide deck, a database file, or other type of digital data structure capable of representing the information in a case summary or settlement demand letter.

FIGS. 3A and 3B illustrate examples of graphical user interfaces in which informational prompts may be presented and responded to by a user. For instance, FIG. 3A illustrates an embodiment of a graphical user interface 301A. The graphical user interface 301A may include a free form question #1 (302A), along with a corresponding answer block 303A, as well as free form question #2 (302B) with its own corresponding answer block 303B. The ellipses indicate that additional free form or other informational prompts may be used. Within the graphical user interface 301A, the user may read the free form question and respond in the answer block. After the free form questions have been answered (and potentially in response to the answers provided by the user), the underlying computer system may change or update the questions, as shown in FIG. 3B.

Graphical user interface 301B may be the same as or different than graphical user interface 301A of FIG. 3A and may, in some cases, represent an updated version of graphical user interface 301A. Graphical user interface 301B may include multi-option selections 304A and 304B, each of which has a corresponding answer box 305A/305B with its own set of multiple-choice answers. Using this technique, the underlying system may obtain more precise answers to specific questions with the flexibility of returning to more open-ended, free form answers when needed. The graphical user interface 301B may be updated to show true/false questions, yes/no questions, additional free form or multiple-choice questions, or other combinations of questions in order to arrive at a desired answer or combination of answers.

FIG. 3C, illustrates a series of questions and answers provided by the underlying system. For instance, the system may generate an initial statement 313 indicating that it is an artificial intelligence (AI) assistant that is designed to help an accident victim with their case. The initial statement 313 may also include one or more questions, potentially asking for details about a recent accident. The user may, in return, provide their first reply or statement 314 via reply box 315, attempting to answer the question(s) posed in 313. In this example, the user's reply is fairly generic and provides few details. In response, the underlying AI system may ask more specific questions about the date and time of the accident. This natural language back and forth may continue, with the AI system continually modifying its questions based on the quality of responses received from the user, until the AI system has acquired some or all of the details and information related to the accident. At this point, the user may select other UI or application options that may be available via button 311 or may change their profile information, if needed, via button 312. Other buttons and UI elements may also be provided within the UI 310.

In cases where specific types of information are to be solicited from the accident victim, the user interface 310 may be updated to include an answer block 317 with specific items that may be selected. The selected items may provide detailed information related to the accident that may inform how a case summary or how a settlement demand letter are to be crafted. The answer block 317 with selectable options may be provided in response to an overly generic reply (e.g., 316) or may be provided when other forms of ascertaining information are inadequate or cumbersome. After presenting the answer block 317 and after the user has made their selections, the user interface 310 may return to a natural language message thread, such as that shown in FIG. 3C above.

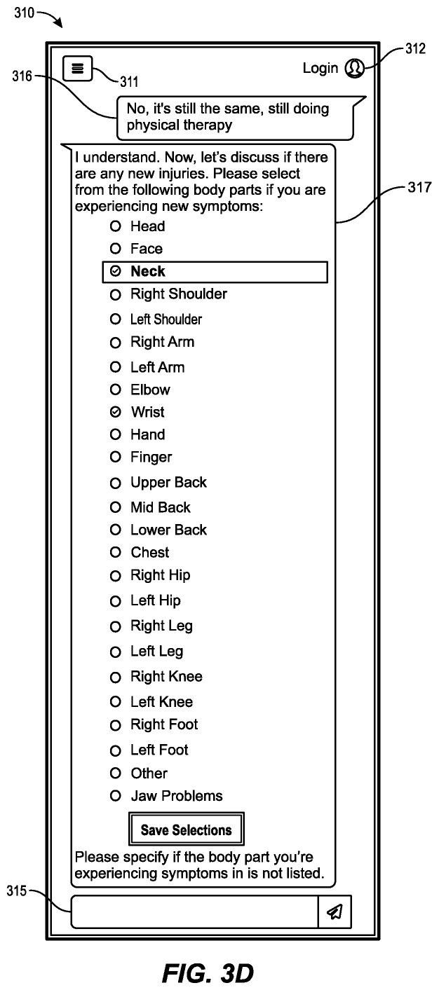

Thus, at least in some embodiments, the informational prompts shown in the graphical user interface may be dynamically generated and may include free-form questions, answer blocks with selectable options, or other types of prompts. In such cases, the user may provide, via the GUI, a free-form answer or a selected answer to the informational prompt. That answer may then be analyzed to obtain additional supporting information related to the informatory elements. This supporting information, as shown in FIG. 4, may include information provided by a user 401, such as an accident victim, an accident witness, an attorney representing the accident victim, or other user. The user 401 may provide different types of supporting information 402 including pictures or videos 403 related to the accident, emails or text messages 404 related to the accident, health records 405 of the accident victim, financial records 406 of the accident victim, or other types of data that may be stored on a decentralized information exchange 407 including health information records.

In some cases, the user 401 may select one or more of the selectable options in a dynamically generated prompt in a graphical user interface. The selected options may be analyzed to obtain additional supporting information 402 related to the informatory elements associated with the event. In some cases, the user 401 may be a party involved in an accident and/or a witness to the accident. That user 401, in addition to answers provided in response to various prompts, may additionally provide, as supporting information 402, data structures in a local data store owned by the user (e.g., stored in the user's phone or other electronic device), data structures that include personal healthcare records (PHRs), data structures that include financial records, and/or data structures stored in a decentralized Health Information Exchange.

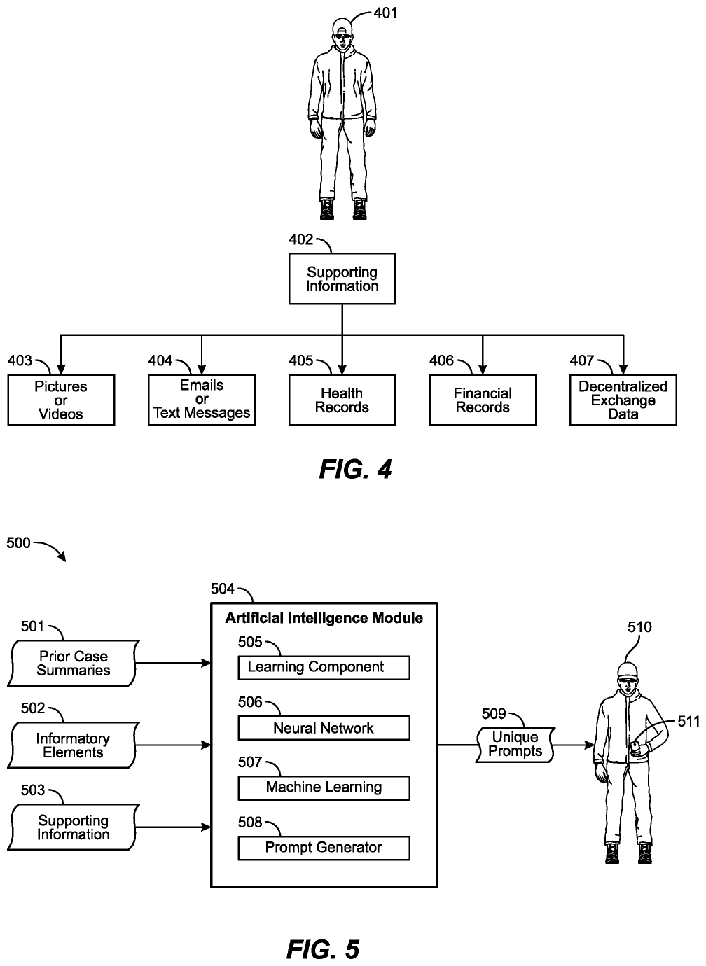

In some cases, informational prompts presented to the user may be generated dynamically and may be unique to the event in which the user was involved. In some scenarios, the dynamically generated informational prompts may be generated using artificial intelligence. Indeed, as shown in embodiment 500 of FIG. 5, in some cases, artificial intelligence modules or algorithms may be trained to determine which informatory elements related to the event meet a minimum significance threshold. For example, artificial intelligence module 504 may access one or more prior case summaries 501. These prior case summaries 501 may represent different fact patterns, different accident victims, and/or different settlement demands. The case summaries 501 may also include the amount demanded or requested in settlement and the ultimate amount received in settlement. Thus, the artificial intelligence module 504 may determine, from the accessed prior case summaries 501, which information led to the optimal settlement results (e.g., the highest amount of recovery or the most comprehensive recovery benefits). As part of this analysis, the artificial intelligence module 504 may also access various informational elements 502 related to an event, along with supporting information 503 that corroborates the informational elements 502.

Upon accessing this information, the artificial intelligence module 504 may be configured to learn (e.g., using learning component 505) from the prior case summaries 501 how to generate prompts that would result in the optimal delivery of facts and information in a new case summary that is to be generated. The artificial intelligence module 504 may implement a neural network 506 and/or a machine learning module 507 to assign weights to different types of informational elements 502 or assign weights to different types of supporting information 503. These weights, in turn, may be used to generate and assign a level of significance to each type of information, noting that some information may be more persuasive or may be more pertinent or may be more valuable for the insurance company (or other entities) to know when considering a case summary or settlement demand letter. The artificial intelligence module 504 may then determine which informational elements 502 meet a minimum threshold significance level and may generate prompts to elicit those types of information. In some embodiments, the underlying system may train a machine learning model to determine, for the event, which informatory elements related to the event meet at least the minimum significance threshold. This training may be carried out by iteratively presenting and analyzing positive examples of case summaries that resulted in beneficial outcomes for the accident victim.

For example, the prompt generator 508 in the artificial intelligence module 504 may generate prompts that are presented to users in a graphical user interface. The prompts may include different types of questions (e.g., free form, multiple choice, true/false, yes/no, or other types of questions) or may include substantively different questions. Thus, in some cases, the prompt generator 508 may generate prompts asking the same or similar questions using different question types. Or, the prompt generator 508 may ask substantively different questions using the same question type or different question types. As the user provides answers to the informational prompts, the artificial intelligence module 504 may analyze, in real time, the user's replies and may dynamically alter or adjust the question type and/or question substance to properly elicit the desired response (i.e., the response that is weighted the highest and, based on learning, would lead to the most effective case summary).

In some embodiments, the artificial intelligence module 504 may be trained to consider at least two of the informatory elements 502 meeting the minimum significance threshold in different manners. For instance, the artificial intelligence module 504 may be modified to consider multiple different elements that meet the minimum significance threshold in different manners for different events. One event with a certain fact pattern may suggest a certain set of weightings for the informational elements 502, while a different fact pattern may suggest a different set of weightings for the informational elements 502 in order to obtain an optimal (or at least desirable) outcome.

As such, the artificial intelligence algorithms and implementations may be customized and tailored to each event and each case summary or demand letter. Indeed, the artificial intelligence may be modified to operate differently for different types of claims, resulting in different, unique prompts 509 being presented to each user (e.g., to user 510 on mobile device 511) in a user interface. In some cases, the artificial intelligence algorithms may, themselves, be weighted in different manners, or may implement different levels of learning to determine which informational prompts led to the best claim-substantiating data.

The user interface may thus present unique informational prompts 509 that prompt the user (e.g., an attorney or pro se individual) to provide information that is specific to the type of claim being sought or to the type of demands outlined in a case summary. By providing answers to these unique prompts, the user may provide at least some of the many types of data related to a specific event. The user's responses may be stored and associated with corresponding substantiating materials including, for example, medical records. In some cases, similar informational prompts 509 may be implemented with different users in relation to different events. As such, at least some of the prompts may be reused by the prompt generator 508 in other cases.

Upon answering the informational prompts, the systems herein may analyze the user's inputs to determine which types of substantiating materials may be the most beneficial to presenting full substantiated and worthwhile claims. The user may then provide access to substantiating, corroborating materials, whether through medical record portals, or by granting limited phone access to copy pictures, videos, text or email messages, or other similar items, or by granting limited banking access to show processed payments. The embodiments described herein may implement many different data sources when gathering supporting information for a claim, whether local, remote, cloud-based, or otherwise. This supporting information may then be automatically incorporated into the case summary or settlement demand letter as generated by the system.

In at least some cases, the systems herein may present a user interface that allows a user (e.g., 510 of FIG. 5) to select which supporting information is to be included in a case summary or settlement demand letter. Accordingly, in such cases, the user may select, deselect, or add new supporting information to the case summary or demand letter. The output digital data structure may include fully written sentences and paragraphs and may be organized in a manner that logically lays out the user's claim in manner that is designed to achieve optimal results, according to the prior case summaries 501. The case summary may include evidence for arguments and claims made in the summary, at least some of which may have been gathered from multiple different databases. This evidence may include pictures, snapshots of videos, text messages, or emails, bank statements, medical records, or other substantiating information.

The dynamically generated informational prompts described above may provide a consistent interview process across different accident victims and fact scenarios. The informational prompts may be designed to ask pertinent questions that will lead to an optimal monetary recovery and will lead the user to provide the most pertinent substantiating information. At least in some cases, the user's answers to these informational prompts 509 may then populate the general damages section of the case summary or demand letter. By going through the unique informational prompts 509, the user may be educated on which types of information are most helpful to provide and which types of information will truly bolster their claim for damages. Upon answering each of the prompts, the underlying system may incorporate at least some supporting information into the case summary. In some embodiments, the user interface may allow the user to select which supporting information is to be included in the case summary. In some cases, the supporting information includes pictures, videos, text messages, email messages, health records, or other supporting information. In some cases, the supporting information may be accessed from a mobile electronic device associated with the user.

A system corresponding to the above-described method may be provided that includes: at least one physical processor and physical memory comprising computer-executable instructions that, when executed by the physical processor, cause the physical processor to: access various portions of data related to an event in which a user was involved, determine, for the event, which informatory elements related to the event meet at least a minimum significance threshold, present to the user, via a graphical user interface, one or more informational prompts associated with the event to obtain supporting information related to those informatory elements associated with the event that were determined to meet the minimum significance threshold, and generate a digital data structure that includes at least a portion of dynamically written language related to the event and further includes at least a portion of the supporting information received from the user in response to the informational prompts.

A non-transitory computer-readable medium corresponding to the above-described method may also be provided that includes one or more computer-executable instructions that, when executed by at least one processor of a computing device, cause the computing device to: access various portions of data related to an event in which a user was involved, determine, for the event, which informatory elements related to the event meet at least a minimum significance threshold, present to the user, via a graphical user interface, one or more informational prompts associated with the event to obtain supporting information related to those informatory elements associated with the event that were determined to meet the minimum significance threshold, and generate a digital data structure that includes at least a portion of dynamically written language related to the event and further includes at least a portion of the supporting information received from the user in response to the informational prompts.

As noted above, in at least some embodiments, the systems herein may implement artificial intelligence when performing one or more of the above method steps. Such artificial intelligence may include deep learning algorithms, machine learning algorithms, neural networks, or other similar systems. In addition to implementing such systems, at least some of the embodiments herein may modify or make improvements to these algorithms. For instance, in cases where a unique questionnaire is implemented that is unique to a specific event, that automated questionnaire may be generated using artificial intelligence (AI). Moreover, the AI may be modified to operate differently for different types of claims, resulting in different questions being presented to the user. For instance, the AI algorithms may be weighted in different manners, or may implement different levels of deep learning to determine which interview questions lead to the best claim-substantiating data. In another example, the AI may be modified to consider multiple different elements that meet the minimum significance threshold in different manners for different events. As such, the AI may be customized and tailored to each event and each demand letter.

As noted above, at least some of the embodiments described herein may train and/or implement a machine learning model. In some cases, the systems herein may be configured to train a neural network to perform any or all of these steps. The systems herein may implement and/or incorporate a machine learning module that includes various ML-related components. These components may include a machine learning (ML) processor, an inferential model, a feedback implementation module, a prediction module, and/or a neural network. Each of these components may be configured to perform different functions with respect to training and/or implementing a machine learning model. The ML processor, for example, may be a dedicated, special-purpose processor with logic and circuitry designed to perform machine learning. The ML processor may work in tandem with the feedback implementation module to access data and use feedback to train an ML model. For instance, the ML processor may access one or more different training data sets. The ML processor and/or the feedback implementation module may use these training data sets to iterate through positive and negative samples and improve the ML model over time.

In some cases, the machine learning module may include an inferential model. As used herein, the term “inferential model” may refer to purely statistical models, purely machine learning models, or any combination of statistical and machine learning models. Such inferential models may include neural networks such as recurrent neural networks. In some embodiments, the recurrent neural network may be a long short-term memory (LSTM) neural network. Such recurrent neural networks are not limited to LSTM neural networks and may have any other suitable architecture. For example, in some embodiments, the neural network may be a fully recurrent neural network, a gated recurrent neural network, a recursive neural network, a Hopfield neural network, an associative memory neural network, an Elman neural network, a Jordan neural network, an echo state neural network, a second order recurrent neural network, and/or any other suitable type of recurrent neural network. In other embodiments, neural networks that are not recurrent neural networks may be used. For example, deep neural networks, convolutional neural networks, and/or feedforward neural networks, may be used. In some implementations, the inferential model may be an unsupervised machine learning model, e.g., where previous data (on which the inferential model was previously trained) is not required.

At least some of the embodiments described herein may include training a neural network to identify data dependencies, identify which information from various data sources is to be altered to lead to a desired outcome, or how to alter the information to lead to a desired outcome. In some embodiments, the systems described herein may include a neural network that is trained to identify how information is to be altered using different types of data and associated data dependencies. For example, the embodiments herein may use a feed-forward neural network. In some embodiments, some or all of the neural network training may happen offline. Additionally or alternatively, some of the training may happen online. In some examples, offline development may include feature and model development, training, and/or test and evaluation.

In one embodiment, a repository that includes data about past data accessed and past data alterations may supply the training and/or testing data. In one example, when the underlying system had accessed different types of data from different data sources, the system may determine which alterations to identify based on data from a feature repository and/or an online recommendation model that may be informed by the results of offline development. In one embodiment, the output of the machine learning model may include a collection of vectors of floats, where each vector represents a data source and each float within the vector represents the probability that a specified data alteration will be identified. In some embodiments, the recent history of a data source may be weighted higher than older history data. For example, if a data source had repeatedly provided relevant data that resulted in relevant operational steps, the ML model may determine that the probability of that data source providing relevant data in the future is higher than for other data sources.

Once the machine learning model has been trained, the ML model may be used to identify which data is to be altered and how that data is to be altered based on multiple different data sets. In some embodiments, the machine learning model that makes these determinations may be hosted on different cloud-based distributed processors (e.g., ML processors) configured to perform the identification in real time or substantially in real time. Such cloud-based distributed processors may be dynamically added, in real time, to the process of identifying data alterations. These cloud-based distributed processors may work in tandem with the prediction module to generate outcome predictions, according to the various data inputs. These predictions may identify potential outcomes that would result from the identified data alterations. The predictions output by the prediction module may include associated probabilities of occurrence for each prediction. The prediction module may be part of a trained machine learning model that may be implemented using the ML processor. In some embodiments, various components of the machine learning module may test the accuracy of the trained machine learning model using, for example, proportion estimation. This proportion estimation may result in feedback that, in turn, may be used by the feedback implementation module in a feedback loop to improve the ML model and train the model with greater accuracy.

Embodiments described herein may implement various types of computing systems. These computing systems are now increasingly taking a wide variety of forms. Computing systems may, for example, be handheld devices such as smartphones or feature phones, appliances, laptop computers, wearable devices, desktop computers, mainframes, distributed computing systems, or even devices that have not conventionally been considered a computing system. In this description and in the claims, the term “computing system” is defined broadly as including any device or system (or combination thereof) that includes at least one physical and tangible processor, and a physical and tangible memory capable of having thereon computer-executable instructions that may be executed by the processor. A computing system may be distributed over a network environment and may include multiple constituent computing systems.

Computing systems typically include at least one processing unit and memory. The memory may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well. As used herein, the term “executable module” or “executable component” can refer to software objects, routines, or methods that may be executed on the computing system. The different components, modules, engines, and services described herein may be implemented as objects or processes that execute on the computing system (e.g., as separate threads).

In the description that follows, embodiments are described with reference to acts that are performed by one or more computing systems. If such acts are implemented in software, one or more processors of the associated computing system that performs the act direct the operation of the computing system in response to having executed computer-executable instructions. For example, such computer-executable instructions may be embodied on one or more computer-readable media that form a computer program product. An example of such an operation involves the manipulation of data. The computer-executable instructions (and the manipulated data) may be stored in the memory of the computing system. Computing system may also contain communication channels that allow the computing system to communicate with other message processors over a wired or wireless network.

Embodiments described herein may comprise or utilize a special-purpose or general-purpose computer system that includes computer hardware, such as, for example, one or more processors and system memory, as discussed in greater detail below. The system memory may be included within the overall memory. The system memory may also be referred to as “main memory” and includes memory locations that are addressable by at least one processing unit over a memory bus in which case the address location is asserted on the memory bus itself. System memory has been traditionally volatile, but the principles described herein also apply in circumstances in which the system memory is partially, or even fully, non-volatile.

Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical hardware storage media that store computer-executable instructions and/or data structures. Physical hardware storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the principles described herein may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

Still further, system architectures described herein can include a plurality of independent components that each contribute to the functionality of the system as a whole. This modularity allows for increased flexibility when approaching issues of platform scalability and, to this end, provides a variety of advantages. System complexity and growth can be managed more easily through the use of smaller-scale parts with limited functional scope. Platform fault tolerance is enhanced through the use of these loosely coupled modules. Individual components can be grown incrementally as business needs dictate. Modular development also translates to decreased time to market for new functionality. New functionality can be added or subtracted without impacting the core system.

In some cases, the computer system may include a communications module that communicates with other computing systems. The communications module may include any wired or wireless communication means that can receive and/or transmit data to or from other computing systems. The communications module may be configured to interact with databases, mobile computing devices (such as mobile phones or tablets), embedded or other types of computing systems.

The concepts and features described herein may be embodied in other specific forms without departing from their spirit or descriptive characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

We claim:

1. A computer-implemented method comprising:
accessing one or more portions of data related to an event in which a user was involved;
determining, for the event, which informatory elements related to the event meet at least a minimum significance threshold;
presenting to the user, via a graphical user interface, one or more informational prompts associated with the event to obtain supporting information related to those informatory elements associated with the event that were determined to meet the minimum significance threshold; and
generating a digital data structure that includes at least a portion of dynamically written language related to the event and further includes at least a portion of the supporting information received from the user in response to the informational prompts.

2. The computer-implemented method of claim 1, wherein the informational prompts are dynamically generated and include at least one free-form answer prompt.

3. The computer-implemented method of claim 2, wherein the user provides a free-form answer to the free-form answer prompt that is analyzed to obtain additional supporting information related to the informatory elements.

4. The computer-implemented method of claim 1, wherein the informational prompts are dynamically generated and include at least one prompt that has multiple selectable options that are selectable via the graphical user interface.

5. The computer-implemented method of claim 4, wherein the user selects at least one of the selectable options in the dynamically generated prompt, and wherein the selected options are analyzed to obtain additional supporting information related to the informatory elements.

6. The computer-implemented method of claim 1, wherein the user comprises at least one of a party involved in an accident or a parent or guardian of a party involved in an accident.

7. The computer-implemented method of claim 1, wherein the user additionally provides, as supporting information, at least one of: data structures in a local data store owned by the user, data structures that include personal healthcare records (PHRs), data structures that include financial records, or data structures stored in a decentralized Health Information Exchange.

8. The computer-implemented method of claim 1, wherein the informational prompts presented to the user are generated dynamically and are unique to the event in which the user was involved.

9. The computer-implemented method of claim 8, wherein the dynamically generated informational prompts are generated using artificial intelligence.

10. The computer-implemented method of claim 9, wherein the artificial intelligence is trained to determine which informatory elements related to the event meet the minimum significance threshold.

11. The computer-implemented method of claim 9, wherein the artificial intelligence is trained to alter the informational prompts presented to the user in the graphical user interface based on which supporting information is provided by the user.

12. The computer-implemented method of claim 1, further comprising training a machine learning model to determine, for the event, which informatory elements related to the event meet at least the minimum significance threshold.

13. A system comprising:
at least one physical processor; and
physical memory comprising computer-executable instructions that, when executed by the physical processor, cause the physical processor to:
access one or more portions of data related to an event in which a user was involved;
determine, for the event, which informatory elements related to the event meet at least a minimum significance threshold;
present to the user, via a graphical user interface, one or more informational prompts associated with the event to obtain supporting information related to those informatory elements associated with the event that were determined to meet the minimum significance threshold; and
generate a digital data structure that includes at least a portion of dynamically written language related to the event and further includes at least a portion of the supporting information received from the user in response to the informational prompts.

14. The system of claim 13, wherein the digital data structure comprises a case summary that includes an analysis of the case.

15. The system of claim 14, wherein at least a portion of the supporting information is incorporated into the case summary.

16. The system of claim 15, wherein the user interface allows the user to select which supporting information is to be included in the case summary.

17. The system of claim 13, wherein the supporting information includes at least one of pictures, videos, text messages, email messages, or health records.

18. The system of claim 17, wherein the supporting information is accessed from a mobile electronic device associated with the user.

19. The system of claim 17, further comprising training a machine learning model to determine, for the event, which informatory elements related to the event meet at least the minimum significance threshold.

20. A non-transitory computer-readable medium comprising one or more computer-executable instructions that, when executed by at least one processor of a computing device, cause the computing device to:
access one or more portions of data related to an event in which a user was involved;
determine, for the event, which informatory elements related to the event meet at least a minimum significance threshold;
present to the user, via a graphical user interface, one or more informational prompts associated with the event to obtain supporting information related to those informatory elements associated with the event that were determined to meet the minimum significance threshold; and
generate a digital data structure that includes at least a portion of dynamically written language related to the event and further includes at least a portion of the supporting information received from the user in response to the informational prompts.
